(19) United States
(12) Patent Application Publication
(10) Pub. No.: US 2014/0301953 A1
(43) Pub. Date: Oct. 9, 2014

(54) ALLERGENS FROM INSECTS

(71) Applicants: LABOR LAUPENECK AG, Bern (CH); UNIVERSITAT BERN, Bern (CH)

(72) Inventors: Beat Bigler, Bern (CH); Andrew Hemphill, Bern (CH); Joachim Muller, Zofingen (CH)

(21) Appl. No.: 14/351,198

(22) PCT Filed: Oct. 5, 2012
(86) PCT No.: PCT/EP2012/069683

§ 371 Date: Apr. 11, 2014

(30) Foreign Application Priority Data
Oct. 11, 2011 (EP) 11184737.2

(51) Int. Cl. A61K 49/00; G01N 33/68; A61K 39/35

(52) U.S. Cl. CPC A61K 49/0006; A61K 39/35; G01N 33/6854
USPC 424/9.81; 424/275.1; 435/7.92

(57) ABSTRACT

The invention relates to test kits for the determination of allergic dermatitis caused by insect bites in an animal such as horses, and to compositions and methods for desensitization. It was found that a culture supernatant or a cell extract of Sf21 insect cells is suitable for reliable test kits and for compositions for desensitization.

### FIELD OF THE INVENTION

The invention relates to test kits and methods for the determination of allergic dermatitis caused by insect bites in an animal and to compositions and methods for desensitization.

### BACKGROUND ART

Insect bite hypersensitivity (IBH) is an allergic dermatitis of horses caused by IgE-mediated reactions to bites of Culicoides and sometimes Simulium spp. Horses suffering from IBH cannot be used for horse riding in the summer. Allergic animals often exhibit massive skin alterations at the feeding sites of female Culicoides, including the mane, tail region and ventral midline. Alterations include papules, broken hair, alopecia, crusts and lichenification of the skin. Secondary alterations caused by scratching can render the situation even more severe. Prophylactic treatments include avoidance of exposure of the horses to the insects by stabling, use of blankets, and use of repellents such as pyrethroide. Symptomatic treatment of affected animals is done with corticosteroids.

Several studies indicate that IBH is caused by a Type-I hypersensitivity reaction (van der Haegen A. et al., Equine Vet J. 2001; 33:699-706; Wilson A. D. et al., Equine Vet J. 2001; 33:707-713). Type-I hypersensitivities are characterized by an initial contact to the allergen, resulting in activation of B-cells and production of allergen-specific IgE antibodies. These antibodies are subsequently bound by high-affinity FCεRI receptor on cell membranes of mast cells and basophils, a process that is called sensitization. The IgE-sensitized cells can be stimulated at each successive contact with allergen, resulting in release of preformed inflammatory mediators such as histamine, and induction of synthesis and release of leukotrienes, prostaglandins and cytokines, a combination of which initiates the inflammatory response and maintains the production of allergen-specific IgE.

The formal proof that IgE directed against Culicoides allergens mediates the classical Type-I allergy in horses and plays a major role in the pathogenesis of IBH comes from two major studies. Wagner B. et al., Vet Res. 2006; 37:813-825, show that in IBH, IgE binds to high-affinity receptors on mast cells, and that this interaction plays a key role in allergic inflammatory responses induced by mast cell degranulation. Furthermore, IgGT can bind to mast cells and might contribute to clinical allergy. Hellberg W. et al., Vet Immunol Immunopathol. 2006; 113:99-112, show that besides IgE also IgGa and IgGT, but not IgGb antibodies, bind to distinct bands on salivary gland extracts of Culicoides nubeculosus.

The diagnosis of IBH includes intradermal testing and detection of allergen-specific IgE by enzyme linked immunosorbent assay (ELISA). Whole insect extracts from Culicoides and Simulium are mostly used as antigens for these assays, with obvious drawbacks such as high production costs, short shelf life, and cross reactivities between species. In all likelihood, the allergens causing IBH are salivary gland proteins from these insects.

### SUMMARY OF THE INVENTION

The invention relates to test kits for determining if an animal is susceptible to or has allergic dermatitis caused by insect bites, said kit comprising:
(a) a culture supernatant or a cell extract of Sf21 insect cells; and
(b) means for determining if body fluids of said animal form immunocomplexes with the culture supernatant or cell extract of Sf21 insect cells.

Furthermore the invention relates to a method of determination if an animal is susceptible to or has allergic dermatitis caused by insect bites by measuring the presence of antibodies indicative of allergic dermatitis in a body fluid of said animal, said method comprising:
(a) contacting a culture supernatant or a cell extract of Sf21 insect cells with a body fluid from said animal under conditions sufficient for formation of an immunocomplex between said culture supernatant or a cell extract and antibodies, if present, in said body fluid; and
(b) determining the amount of immunocomplex formed, wherein formation of said immunocomplex indicates that said animal is susceptible to or has allergic dermatitis.

Likewise the invention relates to a method of determination if an animal is susceptible to or has allergic dermatitis caused by insect bites by measuring the cellular response indicative of allergic dermatitis of said animal, said method comprising:
(a) subcutaneously injecting a dilution series of a culture supernatant or a cell extract of Sf21 insect cells;
(b) determining the amount of inflammatory response around the injection site, wherein the degree of inflammatory response indicates that said animal is susceptible to or has allergic dermatitis.

Furthermore the invention relates a therapeutic composition for treating allergic dermatitis in an animal and for desensitizing an animal to allergic dermatitis comprising a culture supernatant or a cell extract of Sf21 insect cells, and to a method of desensitizing an animal to allergic dermatitis.

### BRIEF DESCRIPTION OF THE FIGURES

FIG. 1. ELISA with recombinant Cn allergens (CnAll) expressed in E. coli.

A: Wells of a 96-well-plate were coated with His-tag-purified recombinant CnAll (1 μg per well) and probed with three different sera from horses previously tested as positive (Pos; black bars) and with three tested as negative (Neg; white bars).
SPI: serine protease inhibitor; D7: D7-protein; Cyspep: cysteine peptidase; Hyal: hyaluronidase; Ag5-Fam: Ag5-family protein; Mal: maltase (see Table 1); mA₄₀₅: milli-absorption at 405 nm.
B: Wells of a 96-well-plate were coated with His-tag-purified recombinant maltase, hyaluronidase (0.5 μg per well each), cysteine peptidase and Ag5-family protein (0.25 μg per well each) and probed with 44 sera from horses previously tested as positive (pos) and with 44 sera from horses previously tested as negative (neg). The dashed line indicates the cut-off.

FIG. 2. Western blot demonstrating the recognition of recombinant Cn allergens expressed in Sf21 cells by IgE from a positive horse (1.4 kU/l). M, size marker with sizes as indicated; 1, crude extract from Culicoides nubeculosus (10 μg), 2-9, crude extracts from Sf21 cells (all 5 μg). 2, Sf21 control; 3, Gus (neg. control); 4, serine protease inhibitor; 5, D7-protein; 6, cysteine peptidase; 7, Ag5-family protein; 8, hyaluronidase; 9, maltase. 6, 8 and 9 are highlighted.

FIG. 3. Western blot demonstrating the differential recognition of allergens present in extracts of Sf21 cells by IgE from a positive horse (1.4 kU/l) versus a negative horse (0.35 kU/l). M, size marker; 1, serine protease inhibitor; 2, D7-protein; 3, cysteine peptidase; 4, Ag5-family protein.

FIG. 4.

A: Western blot demonstrating the differential recognition of allergens present in medium from Sf21 cells secreting recombinant maltase (M) or from control cells (C) by IgE in a serum pool from a positive horses (pos) versus a pool from negative horses (neg).
B: ELISA with the same serum pools (black bars, positive sera; white bars, negative sera). Wells of a 96-well-plate were coated with medium from insect cells expressing maltase or with medium from control cells (1 μg per well).

FIG. 5. Intradermal skin test, with medium of Sf21 cells without (1-3; 9-10) or with C. nubeculosus maltase (4-6). The amounts of injected protein were: 5 ng (1, 4), 2.5 ng (2, 5, 9), or 1 ng (3, 6, 10). Negative (7, 11) and positive (8, 12) controls were included.

A: IBH positive horse, B: IBH negative horse.
A coin of diameter 22 mm is shown for size comparison.

FIG. 6. Development of IgE titers in serum samples from a positive horse before (b) and after (a) a six-week-desensitization with medium of Sf21 cells.

A-D correspond to allergens from biting insects, E and F from mites, G to I from pollen. kU/L: allergen specific IgE titer is given by the recognition of horse IgE by the monoclonal mouse anti-horse-IgE antibody in a standard reaction.

### DETAILED DESCRIPTION OF THE INVENTION

The invention relates to test kits for the determination of allergic dermatitis caused by insect bites and to compositions and methods for desensitization.

In particular the invention relates to such test kits for the determination of insect bite hypersensitivity (IBH) in animals. Animals considered are domestic animals, in particular horses. “Animals” also includes humans, in particular humans with insect-bite allergy.

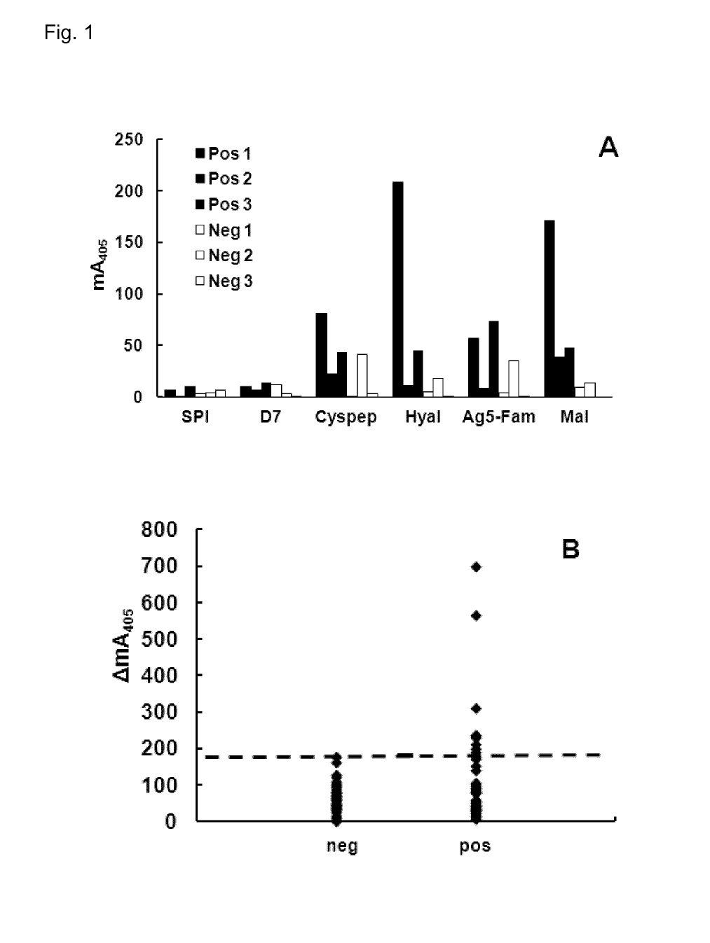

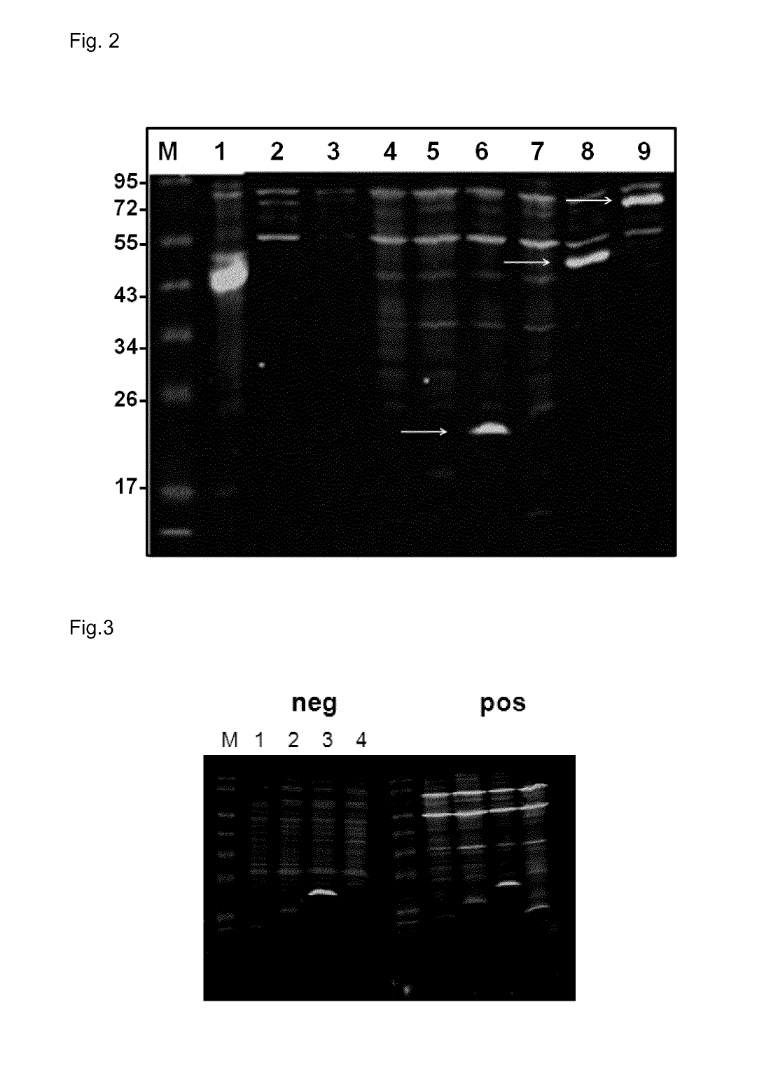

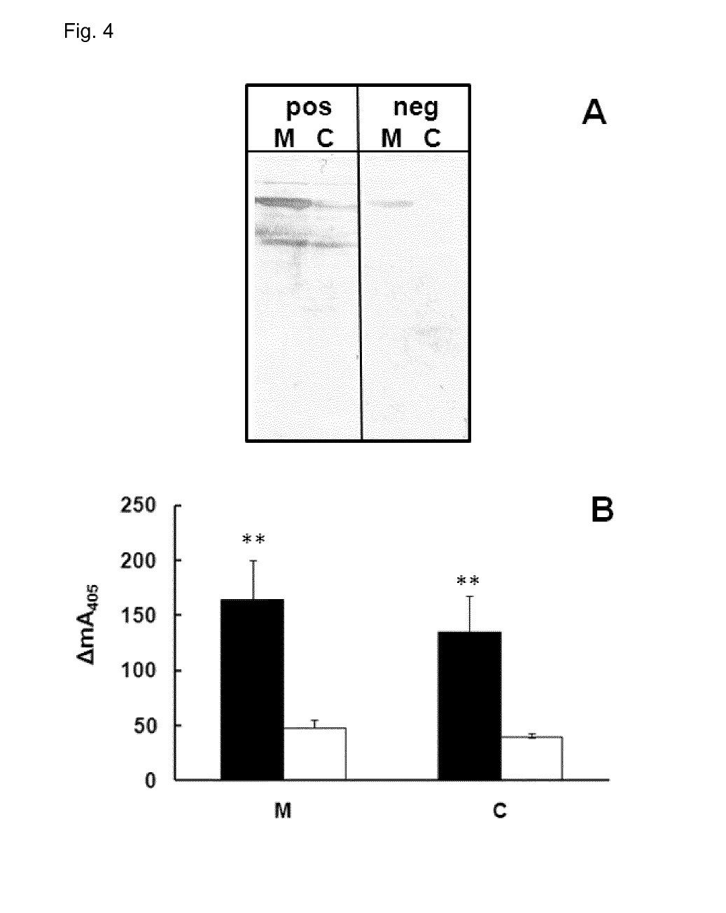

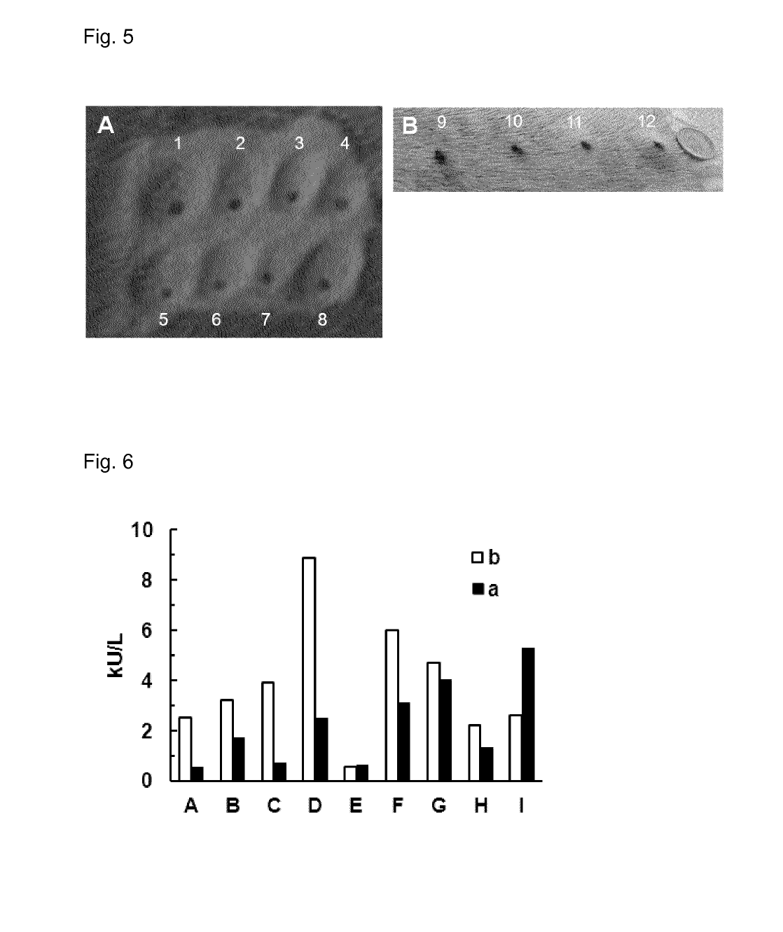

In order to investigate whether a panel of known Culicoides nubeculosus salivary glands proteins allowed to discriminate between IBH positive and negative horses, ELISA plates were coated with recombinant proteins (1 μg/well) from a cDNA library (Russell C. L. et al., Insect Mol Biol. 2009; 18:383-393, Table 1, Cn allergens), and assays were performed with three sera previously tested as positive and three tested as negative using a commercial kit. Maltase allowed the best differentiation, all positive sera giving higher values than the negative ones. With all other proteins, serum from the negative horse 0012 gave higher results than the positive horse 0013. With respect to the overall signal strength, maltase and hyaluronidase gave high, cysteine peptidase and Ag5-family protein intermediate results. The Kunitz inhibitor and the D7 protein allowed no discrimination (FIG. 1A). Based on these results, ELISA plates were coated with maltase, hyaluronidase (0.5 μg/well each), cysteine peptidase and Ag5-family protein (0.25 μg/well each), and assays were performed with 88 sera, 44 previously classified as IBH positive, 44 as negative. A cut-off was calculated as indicated. 10 sera were above this cut-off value (FIG. 1B).

TABLE 1
List of recombinant allergens from C. nubeculosus (Cn allergens)
The number of known allergens of the respective protein family is
given as published by http://www.meduniwien.ac.at/allergens/allfam/
(Summer 2010).

| | | | Number |
|---|---|---|---|
| | | Size | of related |
| Allergen | Accession No. | (kDa) | allergens |
| Amylase/maltase | ACM40914 | 75 | 8 |
| Hyaluronidase | ACM40915.1 | 50 | 5 |
| Cys-peptidase (papain-like) | ACM40897.1 | 45 | 10 |
| Ca²⁺-serine protease (Ag5-family; | ACM40909.1 | 27 | 22 |
| PR-1-like) | | | |
| D7-protein (pheromone/odorant | ACM40878.1 | 14 | 3 |
| binding domain) | | | |
| Serin protease inhibitor (Kunitz) | ACM40879.1 | 13 | 6 |

In a next series of experiments, immunoblottings were performed with the Cn allergens expressed in insect cells and with maltase canonically secreted to the medium mediated by an HBM signal peptide. When the proteins were expressed intracellularly, only cysteine peptidase, hyaluronidase and maltase were recognized by IgE from a serum previously identified as positive. Hyaluronidase and maltase ran at or above their expected molecular weights, namely 50 and 70 kDa, respectively, cysteine peptidase gave a signal at around half its expected size, namely around 20 kDa. This was reproducible and could also been observed upon decoration with anti-His-tag antibodies. Interestingly, besides the recombinant proteins, other bands also present in control extracts were recognized as well (FIG. 2).

Since the constitutive proteins from Sf21 cells are also differently recognized by positive and negative sera, a Western Blot with proteins from insect cells expressing cysteine peptidase (recognized by horse IgE) and with proteins from three other cell lines expressing SPI, D7, and Ag5-family protein (not recognized) was performed. The blots were incubated with sera from a positive and from a negative horse. In the case of the positive serum, not only the recombinant cysteine peptidase gave a stronger signal, but also other constitutively expressed proteins (FIG. 3). Similar results were obtained when protein from the medium was used instead of cell extracts.

The Cn allergen maltase could be expressed as a canonically secreted protein in Sf21 cells and was recognized by IgE from IBH positive horses (FIG. 4 A). Interestingly, also in medium from control cells, bands were recognized by positive and to a much lesser extent by negative sera. Hence the medium from control insect cells can also be used as an antigen source for ELISA. Wells were coated either with medium protein from Sf21 secreting maltase or with medium protein from Sf21 cells transformed with an empty bacmid, and tested with sera identified as positive or negative in the screen with Cn allergens produced in E. coli as described before (see FIG. 1 B). Both ELISAs allowed to discriminate between positive and negative horses (t-tests, p<0.01). There were, however, no significant differences between medium protein from maltase expressing and non-expressing cells (FIG. 4 B).

It is known that salivary gland proteins from C. nubeculosus expressed in E. coli can be used as antigens in diagnostic test kits allowing the discrimination between IBH positive and negative horses (Schaffartzik A. et al., Vet Immunol Immunopathol. 2011; 139:200-209). In the present invention, it is demonstrated that extracellular proteins from medium from cultivated Sf21 cells give very similar results when they are employed as antigens in immunoblotting and ELISAs. Medium from cells expressing maltase, an allergen with a strong response in the ELISA described above, gives only slightly higher signals in ELISAs. The majority of the IgE response of IBH horses is directed against common epitopes present on a variety of insect proteins. Sf21 insect cell medium thus provides an inexpensive source of IBH allergens that can be used in diagnostic tests and in a desensitization therapy.

The present invention relates to an assay kit for testing if an animal is susceptible to or has allergic dermatitis, said kit comprising:
(a) a culture supernatant or a cell extract of Sf21 insect cells; and
(b) means for determining if body fluids of said animal form immunocomplexes with the culture supernatant or cell extract of Sf21 insect cells.

A kit of the invention may comprise further components, such as positive and negative samples for comparison, wash and blocking solutions, and standard operating protocols.

Means for determining if body fluids immunocomplexes with culture supernatant or cell extract of Sf21 insect cells are, e.g., test strips, dipsticks, beads or plates, control samples (negative and positive), reagents for detecting immunocomplexes, e.g. labeled secondary antibodies, color reagents for developing bound labels, and the like.

In an alternative embodiment such means may be suitable for performing a skin test, and may comprise syringes and/or apparatus for applying syringes. Determination if body fluids form immunocomplexes is then performed based on observation of the skin near the injection of culture supernatant or cell extract of Sf21 insect cells.

### Examples of Such Kits are:

(1) Fast test kit with insect medium protein. The kit contains a test strip with insect medium protein from a culture supernatant or a cell extract of Sf21 insect cells, a positive and a negative control protein, wash buffer, an anti-IgE-alkaline phosphatase or anti-IgE-horse radish peroxidase conjugate specific for the particular animal IgE or human IgE, a reagent for color development (phosphatase or peroxidase), standard operating protocol.
(2) ELISA plate kit. The kit contains ELISA plates coated with insect cell protein from a culture supernatant or a cell extract of Sf21 insect cells, stock solutions or premix for the preparation of wash and blocking solutions, positive and negative control sera, anti-IgE-conjugate (alkaline phosphatase or horse radish peroxidase), reagent for color development, standard operating protocol.
(3) Kit for intradermal testing. The kit contains a dilution series of insect medium protein from a culture supernatant or a cell extract of Sf21 insect cells in a suitable buffer, positive and negative control solutions, an intradermic syringe, a panel of representative photos for comparison, standard operating protocol.

Furthermore the invention relates to a method of determining if an animal is susceptible to or has allergic dermatitis, such as IBH, by measuring the presence of antibodies indicative of allergic dermatitis, in a body fluid of said animal, comprising:
(a) contacting a culture supernatant or a cell extract of Sf21 insect cells with a body fluid from said animal under conditions sufficient for formation of an immunocomplex between said culture supernatant or cell extract and antibodies, if present, in said body fluid; and
(b) determining the amount of immunocomplex formed, wherein formation of said immunocomplex indicates that said animal is susceptible to or has allergic dermatitis.

Kits (1) or (2) are useful in the mentioned method determining the humoral immunological response.

Typical results are for kit (1): Spots for positive control and insect medium protein in the case of a positive serum, positive control spot only in the case of a negative serum. For kit (2), numerical results are obtained which are interpreted in function of a cutoff value. Values above the cutoff are interpreted as positive, values below as negative. The cutoff is calculated as the mean value of a panel of defined negative sera plus three times the standard deviation of these values.

Furthermore the invention relates to a method of determining if an animal is susceptible to or has allergic dermatitis, such as IBH, by measuring the cellular response indicative of allergic dermatitis of said animal, said method comprising:
(a) subcutaneously injecting a dilution series of a culture supernatant or a cell extract of Sf21 insect cells;
(b) determining the amount of inflammatory response around the injection site, wherein the degree of inflammatory response indicates that said animal is susceptible to or has allergic dermatitis.

Kit (3) is suitable for determination of a cellular response. A dilution series of insect medium protein, a positive and a negative control are injected into the skin. Short term inflammatory reactions around the injection sites are photographed and compared to a typical response pattern. In the case of a positive animal, the threshold value of such reactions is obtained with higher dilutions of the protein as in the case of a negative animal.

A culture supernatant of Sf21 insect cells is obtained, for example, by cultivating Sf21 cells infected or not with a suitable baculovirus strain for five days as adherent cultures in cell culture flasks or as suspension culture. Subsequently the medium is harvested, and centrifuged at 500×g for 10 min at 20° C. in order to remove detached cells. The medium is then dialyzed against ammonium bicarbonate in two steps (1 g/l followed by 0.25 g/l), lyophilized, and stored in convenient aliquots at −20° C. In order to standardize the batches, the protein content is determined by a standard method (e.g. Bradford).

A cell extract of Sf21 insect cells is obtained, for example, by suspending the cellular pellets obtained by the centrifugation described above in an extraction buffer, namely phosphate buffered saline containing 0.5% (v/v) Triton-X-100 and a commercial protease inhibitor cocktail. Extraction is performed by repeated chilling-vortexing cycles followed by centrifugation at 15′000×g for 10 min at 4° C. The extracts are then further processed as described above for the medium.

The present invention relates also to a therapeutic composition for treating allergic dermatitis in an animal comprising a culture supernatant or a cell extract of Sf21 insect cells.

The composition comprises a culture supernatant or a cell extract of Sf21 insect cells prepared as described above.

For desensitization, a suitable amount of a culture supernatant or a cell extract of Sf21 insect cells or the corresponding therapeutic composition is given at the threshold dilution as determined in the intradermal test in the same buffer. The protein mixture is applied, for example, by injection once or twice a day into the mandibular lymph nodes.

The present invention further relates to a method of desensitizing an animal to allergic dermatitis, such as insect bite hypersensitivity, comprising administering to said animal a therapeutic composition comprising a culture supernatant or a cell extract of Sf21 insect cells.

### EXAMPLES

### Horse Sera

Horse sera were collected in glass tubes or tubes with LiHeparin after venous puncture and centrifuged after 20 min. Serum and plasma fractions were collected, frozen and stored at −20° C. All sera were tested for the presence of anti-Culicoides-IgE using a commercial kit:Biocheck GmbH, D-48159 Munster: Polycheck Equus1 05 001 022. Sera with a titer of 1 kU/l and above were regarded as positive. The titer is defined by the recognition of horse IgE titers by a monoclonal mouse anti-horse-IgE antibody in a standard reaction.

### Chemicals

If not otherwise stated, all chemical reagents were from Sigma-Aldrich Chemie GmbH (Buchs, Switzerland).

### Cloning and Recombinant Expression

In order to clone six proteins from C. nubeculosus (Table 1, henceforth referred to as CnAll), RNA was extracted from C. nubeculosus flies using the PeqGold kit according to the manufacturer's instructions. cDNA was prepared by standard methods (Müller J. et al., Antimicrob Agents Chemother. 2007; 51:1979-1986). The CnAll coding sequences were amplified by PCR and cloned into the respective vectors, i e. pETHis151 for expression in E. coli, pFastBac/NT-TOPO for expression as cytosolic products and pFastBac/HBM-TOPO for honey bee mellitin (HBM) signal peptide mediated expression as secreted products in insect cells (all from Invitrogen, Carlsbad, Calif.) according to the manufacturer's instructions.

For expression in insect cells, bacmids were prepared using the Bac-to-Bac-TOPO cloning kit (Invitrogen) as described by the manufacturer. For transfection with the recombinant bacmids, Sf21 cells were grown as adherent cultures in T-75 cm² flasks to exponential phase in Sf-900II serum free medium until exponential growth phase was reached. Then, cells were plated and transfection was performed according to the user manual “Bac-to-Bac TOPO expression system” (Invitrogen) in 6-well-plates. After 5 days, suitable aliquots of the supernatants representing the viral stock P1 were transferred to fresh adherent cultures in T-75 cm² flasks. When the cells started to detach (usually after 5 days), cells were harvested by centrifugation (500×g, 5 min, room temperature), and cells and medium were analyzed separately for the presence of recombinant CnAll. In some experiments, a control bacmid containing E. coli glucuronidase A (provided by Invitrogen) was included.

Immunoassays using Recombinant CnAll

Expression of recombinant proteins in E. coli was induced and proteins were purified as described (Muller J. et al., 2007, loc. cit.) with the difference that all buffers contained 5 M guanidium hydrochloride. Proteins from insect cells were extracted using PBS containing 0.1% Triton-X-100 and 1% of a commercial protease inhibitor cocktail (Halt, Thermo Scientific, Rockland, Ill.). Western blots were performed as described previously (Nillius D. et al., J Antimicrob Chemother. 2011; 66:1029-1035). Enzyme-linked solid-phase immunoassays (ELISA) were performed as described with the only differences that bovine hemoglobin (0.5 g/l) was used instead of milk powder and that a commercial solution of a mouse-anti-horse IgE antibody (Equus) was used in order to allow the comparison with the results from a commercial kit (see above).

### Statistics

Analysis of variance and subsequent pairwise t-tests were performed using the Excel software package (Microsoft, Seattle, Wash.).

### Analysis of Allergic Horses

Out of 56 horses showing positive effects (IgE against crude extract of Sf21 cells) in the allergy test, 43 showed allergen-specific IgE towards several biting insects (e.g. those shown in FIG. 6, A-D). A correlation of Sf21 allergen and allergens from biting insects in the allergic test is obvious.

### Treatment Results

Sixteen allergic horses were desensitized with cell extract of Sf21 cells. One healthy horse served as negative control. 11/16 horses (68.75%) showed a clear clinical improvement. These animals did not show any symptoms or only very week symptoms.

Thirteen horses were tested for allergen-specific IgE and IgG (allergen: cell extract of Sf21 cells). 9/13 horses (69.25%) had a reduction of IgE and at the same time an increase of IgG. In 3/13 horses both IgE and IgG were increased. In 1/13 horse both IgE and IgG were reduced.

1. A test kit for determining if an animal is susceptible to or has allergic dermatitis caused by insect bites, said kit comprising:
(a) a culture supernatant or a cell extract of Sf21 insect cells; and
(b) means for determining if body fluids of said animal form immunocomplexes with the culture supernatant or cell extract of Sf21 insect cells.

2. The test kit according to claim 1 wherein component (a) is a culture supernatant of Sf21 insect cells.

3. The test kit according to claim 1 wherein component (a) is a cell extract of Sf21 insect cells.

4. The test kit according to claim 1 comprising a test strip with insect medium protein from a culture supernatant or a cell extract of Sf21 insect cells, a positive and a negative control protein, wash buffer, an anti-IgE-alkaline phosphatase or anti-IgE-horse radish peroxidase conjugate specific for the particular animal IgE or human IgE, and phosphatase or peroxidase for color development.

5. The test kit according to claim 1 comprising an ELISA plate coated with insect cell protein from a culture supernatant or a cell extract of Sf21 insect cells, stock solutions or premix for the preparation of wash and blocking solutions, positive and negative control sera, an anti-IgE-alkaline phosphatase or anti-IgE-horse radish peroxidase conjugate specific for the particular animal IgE or human IgE, and phosphatase or peroxidase for color development.

6. The test kit according to claim 4 comprising an anti-IgE-alkaline phosphatase conjugate, and phosphatase for color development.

7. The test kit according to claim 4 comprising an anti-IgE-horse radish peroxidase conjugate, and peroxidase for color development.

8. The test kit according to claim 4 wherein the anti-IgE conjugate is specific for equine IgE.

9. The test kit according to claim 1 comprising a dilution series of insect medium protein from a culture supernatant or a cell extract of Sf21 insect cells in buffer solution, positive and negative control solutions, an intradermic syringe, and a panel of representative photos for comparison.

10. A method of determination if an animal is susceptible to or has allergic dermatitis caused by insect bites by measuring the presence of antibodies indicative of allergic dermatitis in a body fluid of said animal, said method comprising:
(a) contacting a culture supernatant or a cell extract of Sf21 insect cells with a body fluid from said animal under conditions sufficient for formation of an immunocomplex between said culture supernatant or a cell extract and antibodies, if present, in said body fluid; and
(b) determining the amount of immunocomplex formed, wherein formation of said immunocomplex indicates that said animal is susceptible to or has allergic dermatitis.

11. A method of determination if an animal is susceptible to or has allergic dermatitis caused by insect bites by measuring the cellular response indicative of allergic dermatitis of said animal, said method comprising:
(a) subcutaneously injecting a dilution series of a culture supernatant or a cell extract of Sf21 insect cells;
(b) determining the amount of inflammatory response around the injection site, wherein the degree of inflammatory response indicates that said animal is susceptible to or has allergic dermatitis.

12. A therapeutic composition for treating allergic dermatitis in an animal comprising a culture supernatant or a cell extract of Sf21 insect cells.

13. A therapeutic composition for desensitizing an animal to allergic dermatitis comprising a culture supernatant or a cell extract of Sf21 insect cells.

14. A method of desensitizing an animal to allergic dermatitis, comprising administering to said animal a therapeutic composition comprising a culture supernatant or a cell extract of Sf21 insect cells.

15. The method of claim 14 wherein the composition is injected into the mandibular lymph nodes.
